(19) United States
(12) Patent Application Publication
(10) Pub. No.: US 2007/0142827 A1
(43) Pub. Date: Jun. 21, 2007

(54) Dynamic laser beam characteristic measurement system for ophthalmic surgery and associated methods

(76) Inventors: Eugene O. Curatu, Orlando, FL (US); Richard TY Olmstead, Oviedo, FL (US)

Correspondence Address: ALCON, IP LEGAL, TB4-8, 6201 SOUTH FREEWAY, FORT WORTH, TX, 76134, US

(21) Appl. No.: 11/313,287

(22) Filed: Dec. 20, 2005

(51) Int. Cl. A61F 9/008

(52) U.S. Cl. 606/10; 606/5

(57) ABSTRACT

A system and method for determining and monitoring a laser ablation volume of a cornea. The method includes sampling a beam of laser shots from a pulsed treatment laser as a reference portion. A fluence distribution of the beam reference portion is measured, and a laser beam characteristic is calculated from the measured fluence distribution. A system for determining and monitoring a laser ablation volume of a cornea includes a beamsplitter that is positioned to split a beam of laser shots from a pulsed treatment laser into a corneal portion and a reference portion. Devices are provided for measuring a fluence distribution of the beam reference portion and for calculating an ablation volume per laser shot from the measured fluence distribution.

### FIELD OF THE INVENTION

The present invention is directed to laser surgery on the eye, and, more particularly, to systems and methods for calculating and monitoring a characteristic of a laser beam in real time of a laser ophthalmic surgical device.

### BACKGROUND OF THE INVENTION

In surgery on the eye, the laser system must be calibrated carefully. For example, in refractive laser surgery, typically using an excimer laser system, the volume of material ablated by each laser shot must be calibrated. It is currently known in the art to perform such a calibration measurement on a synthetic material such as a plastic that is selected for being analogous to the human cornea. Then the volume of material ablated per laser shot is calculated by a diameter of an ablation “crater” produced by a given number of laser shots.

The underlying theory of this method is based on a number of assumptions. (1) The interaction of the plastic material and the laser radiation is independent of the number of laser shots; (2) there is a well-known relationship between the volume per shot in the plastic material used for calibration and the volume per shot in the cornea; and (3) the volume per shot is calculated with the assumption that the excimer laser profile is Gaussian and rotationally symmetric.

Other laser system characteristics also must be calibrated. For example, in ultrafast laser systems it is important to determine any changes to the temporal response throughout a surgical procedure.

It would be beneficial to provide a more accurate system and method for performing calibration on a laser surgical system, such as one that could be used in real time during a surgical procedure.

### SUMMARY OF THE INVENTION

The present invention is directed to a system and method for determining and monitoring a laser ablation volume of a cornea. The method comprises the step of sampling a beam of laser shots from a pulsed treatment laser as a reference portion. A fluence distribution of the beam reference portion is measured, and an ablation volume per laser shot is calculated from the measured fluence distribution. Fluence is defined as the total amount of energy applied per unit area, measured, for example, in joules/cm².

A system for determining and monitoring a laser ablation volume of a cornea comprises a beam sampler such as a beamsplitter that is positioned to split a beam of laser shots from a pulsed treatment laser into an incident portion, for example, for effecting a treatment, and a sampling, or reference, portion. Means are provided for measuring a fluence distribution of the beam sampling portion and for calculating an ablation volume per laser shot from the measured fluence distribution.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWING

FIG. 1A is a system schematic of the ablation and monitoring system of the present invention configured for calibration.

FIG. 1B is a system schematic of the ablation and monitoring system of the present invention configured for surgery.

FIG. 2 is a flowchart of an exemplary embodiment of the method of the present invention.

### DETAILED DESCRIPTION OF THE PREFERRED EMBODIMENTS

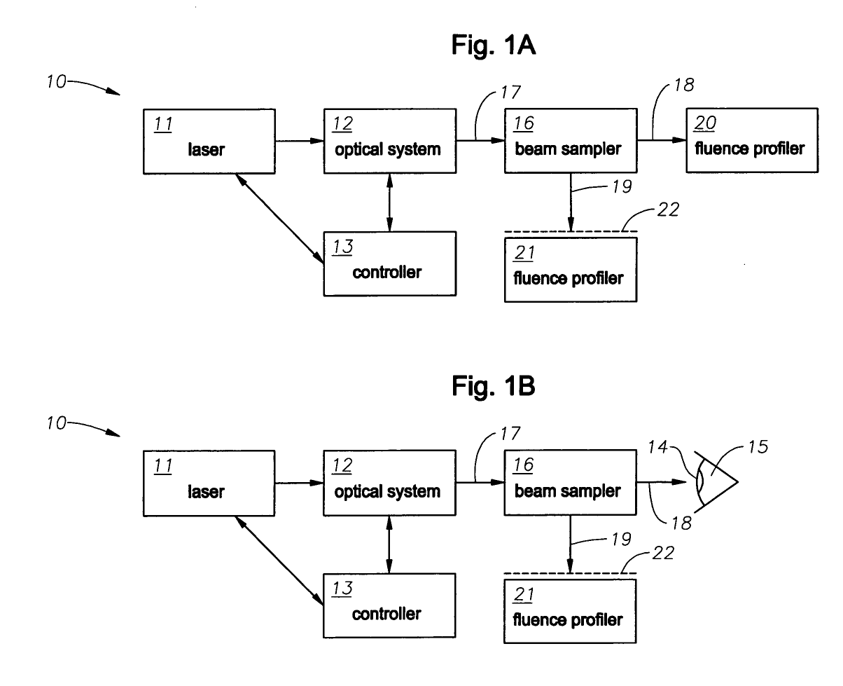

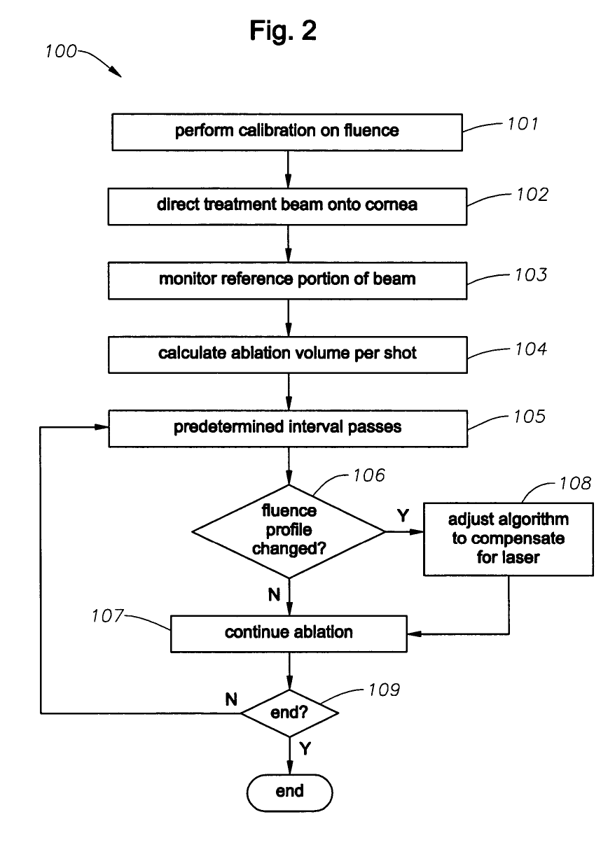

A description of preferred embodiments of the invention will now be presented with reference to FIGS. 1A-2. A system 10 for performing a corneal laser ablation comprises a pulsed treatment laser 11 that can be, for example, an excimer laser (FIGS. 1A, 1B). An optical system 12 is in an optical path of the laser 11. A controller 13 is in signal communication with the optical system 12 for controlling a placement of shots from the treatment laser 11 based upon a predetermined ablation profile. As is known in the art, the ablation profile will typically have been determined previously on a cornea 14 of an eye 15.

A beam sampler such as a beamsplitter 16 is positioned to sample laser shots 17 from the treatment laser 11 into a treatment portion 18 and a reference portion 19. In an exemplary embodiment for corneal refractive surgery, for example, the treatment portion 18 will be incident on the cornea 14, and will be referred to as a “corneal portion.” In a method 100 of the present invention, preferably, a calibration is performed prior to the surgery by determining a fluence distribution of the corneal portion 18 (block 101; FIG. 1A), for example, with the use of a measurement device 20 such as a laser beam fluence profiler or energy meter. The method 100 will be described relative to the exemplary embodiment of corneal refractive surgery, but this is not intended as a limitation on the uses of the invention for other types of laser surgery.

Means for measuring a fluence distribution of the beam reference portion, for example, a laser beam fluence profiler 21, is positioned at a corneal plane 22. During surgery, the beamsplitter 16 directs the corneal portion 18 onto the cornea 14 in a predetermined pattern to achieve the predetermined ablation profile, under direction of the controller 13 (block 102). The ablation profile, however, is based upon assumptions on the volume of corneal tissue ablated per shot (VPS), and it is therefore believed beneficial to provide an accurate and periodic, real-time calibration of this value.

In order to provide such a calibration, the reference portion 19 is monitored by the laser beam fluence profiler 21 (block 103) with the use of the following calculations. If the radiance fluence or radiant exposure F₀ in a particular point of an excimer laser beam spot at the corneal plane, the depth of ablation per pulse can be calculated using Beer's law, which describes the attenuation of light in an optically absorbing medium:
F=F₀·exp[−α·z] (1)
where F₀ is the fluence at the separation surface, a is the absorption coefficient, and F is the fluence at a depth z in the absorbent medium.

Assuming that the ablation threshold fluence is known and the laser fluence at a point (x,y) of the ablation area A, the ablation depth at this point is given by:

$$D ( x , y ) = \frac{1}{α} ln \frac{F ( x , y )}{{F}_{TH}} \qquad ( 2 )$$

The ablation volume per laser pulse, or volume per shot VPS, is given by:

$$\begin{matrix}VPS = {\int }_{A} \int D ( x , y ) \cdot ⅆ x ⅆ y \\ = \frac{1}{a} [ {\int }_{A} \int ln F ( x , y ) \cdot ⅆ x ⅆ y ] - \frac{A \cdot ln {F}_{TH}}{α}\end{matrix} \qquad ( 3 )$$

and A is the pulse ablation area that can be defined by F(x,y)=F_{TH}.

Therefore, a measurement of the fluence distribution of the pulsed excimer laser 11 at the corneal plane 22, and applying Eq. (3) (block 104), an accurate estimate of the volume per shot is obtained.

Preferably the reference calculation is performed at predetermined intervals during a corneal ablation procedure (block 105). If the VPS value is substantially unchanged from the previous measurement (block 106), no change to the ablation profile is made, and the procedure continues (block 107). If the VPS value is changed substantially (block 106), the system is adjusted (block 108) prior to continuing the procedure (block 107). This process continues until the profile is complete (block 109).

Thus is can be seen that a more accurate and real-time calibration can be performed on the volume per shot, without making an assumption as to the beam profile, and also without making an assumption as to the equivalence between the cornea and a synthetic material.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

What is claimed is:

1. A method for determining and monitoring a laser beam characteristic of an ophthalmic treatment laser comprising the steps of:
sampling a beam of laser shots from a pulsed treatment laser as a reference portion;
measuring a fluence distribution of the beam reference portion; and
calculating a laser beam characteristic from the measured fluence distribution.

2. The method recited in claim 1, wherein the fluence distribution is measured at a treatment plane.

3. The method recited in claim 1, wherein the laser beam characteristic comprises an ablation volume per shot calculated as a function of an ablation depth at each point of an ablation area covered by the laser shot.

4. The method recited in claim 3, wherein the ablation volume per shot is calculated by performing an integration over the ablation area of the ablation depth, wherein the ablation depth is calculated as:
$$D ( x , y ) = \frac{1}{α} ln \frac{F ( x , y )}{{F}_{TH}}$$
where α is the absorption coefficient, F is the fluence at a point (x,y), and F_{TH} is the threshold fluence for a cornea.

5. The method recited in claim 4, wherein the integration over the ablation area of the ablation depth equals:
$$\begin{matrix}VPS = {\int }_{A} \int D ( x , y ) \cdot ⅆ x ⅆ y \\ = \frac{1}{a} [ {\int }_{A} \int ln F ( x , y ) \cdot ⅆ x ⅆ y ] - \frac{A \cdot ln {F}_{TH}}{α}\end{matrix}$$
where A is the pulse ablation area defined by F(x,y)=F_{TH}, the threshold fluence for a cornea.

6. The method recited in claim 1, wherein the measuring step comprises using a laser beam fluence profiler.

7. The method recited in claim 1, wherein the sampling step comprises splitting the beam of laser shots into a treatment portion and the reference portion, and further comprising the step of determining a fluence distribution of the treatment portion for calibrating the fluence distribution of the beam reference portion measuring step.

8. The method recited in claim 1, further comprising repeating the measuring and calculating steps at predetermined intervals during an ophthalmic surgical procedure.

9. The method recited in claim 8, further comprising the step of adjusting at the predetermined intervals a treatment protocol being implemented during the procedure based upon the calculated laser beam characteristic.

10. A system for determining and monitoring a laser beam characteristic comprising:
a sampler positioned to sample a beam of laser shots from a pulsed treatment laser as a reference portion;
means for measuring a fluence distribution of the beam reference portion; and
means for calculating a laser beam characteristic from the measured fluence distribution.

11. The system recited in claim 10, wherein the fluence distribution measuring means is positioned at a treatment plane.

12. The system recited in claim 10, wherein the laser beam characteristic comprises an ablation volume per shot calculated as a function of an ablation depth at each point of an ablation area covered by the laser shot.

13. The system recited in claim 12, wherein the ablation volume per shot is calculated by performing an integration over the ablation area of the ablation depth, wherein the ablation depth is calculated as:
$$D ( x , y ) = \frac{1}{α} ln \frac{F ( x , y )}{{F}_{TH}}$$
where α is the absorption coefficient, F is the fluence at a point (x,y), and F_{TH} is the threshold fluence for a cornea.

14. The system recited in claim 13, wherein the integration over the ablation area of the ablation depth equals:
$$\begin{matrix}VPS = {\int }_{A} \int D ( x , y ) \cdot ⅆ x ⅆ y \\ = \frac{1}{a} [ {\int }_{A} \int ln F ( x , y ) \cdot ⅆ x ⅆ y ] - \frac{A \cdot ln {F}_{TH}}{α}\end{matrix}$$
where A is the pulse ablation area defined by F(x,y)=F_{TH}, the threshold fluence for a cornea.

15. The system recited in claim 10, wherein the measuring means comprises a laser beam fluence profiler.

16. The system recited in claim 10, wherein the sampler comprises a beamsplitter for splitting the beam of laser shots into a treatment portion and the reference portion, and further comprising means for determining a fluence distribution of the treatment portion for calibrating the fluence distribution of the beam reference portion fluence measuring means.

17. The system recited in claim 10, further comprising a controller for signaling the measuring and calculating means to perform at predetermined intervals during a an ophthalmic surgical procedure.

18. The system recited in claim 17, further comprising means for adjusting at the predetermined intervals a treatment protocol being implemented during the procedure based upon the calculated laser beam characteristic.

19. A system for performing an ophthalmic surgical procedure comprising:
a pulsed treatment laser;
an optical system in an optical path of the laser;
a controller in signal communication with the optical system for controlling a laser beam characteristic based upon a predetermined treatment protocol;
a sampler positioned to sample a beam of laser shots from the treatment laser as a reference portion;
means for measuring a fluence distribution of the beam reference portion;
means for calculating laser beam characteristic from the measured fluence distribution;
means in communication with the controller for adjusting the treatment protocol based upon the calculated laser beam characteristic.

20. The system recited in claim 21, wherein the fluence distribution measuring means is positioned at a treatment plane.

21. The system recited in claim 19, wherein the laser beam characteristic comprises an ablation volume per shot calculated as a function of an ablation depth at each point of an ablation area covered by the laser shot.

22. The system recited in claim 21, wherein the ablation volume per shot is calculated by performing an integration over the ablation area of the ablation depth wherein the ablation depth is calculated as:
$$D ( x , y ) = \frac{1}{α} ln \frac{F ( x , y )}{{F}_{TH}}$$
where α is the absorption coefficient, F is the fluence at a point (x,y), and F_{TH} is the threshold fluence for a cornea.

23. The system recited in claim 22, wherein the integration over the ablation area of the ablation depth equals:
$$\begin{matrix}VPS = {\int }_{A} \int D ( x , y ) \cdot ⅆ x ⅆ y \\ = \frac{1}{a} [ {\int }_{A} \int ln F ( x , y ) \cdot ⅆ x ⅆ y ] - \frac{A \cdot ln {F}_{TH}}{α}\end{matrix}$$
where A is the pulse ablation area defined by F(x,y)=F_{TH}, the threshold fluence for a cornea.

24. The system recited in claim 19, wherein the measuring means comprises a laser beam fluence profiler.

25. The system recited in claim 19, wherein the sampler comprises a beamsplitter for splitting the beam of laser shots into a treatment portion and the reference portion, and further comprising means for determining a fluence distribution of the treatment portion for calibrating the fluence distribution of the beam reference portion fluence measuring means.

26. The system recited in claim 19, further comprising a controller for signaling the measuring and calculating means to perform at predetermined intervals during an ophthalmic surgical procedure.

27. The system recited in claim 26, wherein the adjusting means operates at the predetermined intervals to adjust the treatment protocol being implemented during the procedure based upon the calculated laser beam characteristic.
